(19) United States
(12) Patent Application Publication
(10) Pub. No.: US 2026/0325719 A1
(43) Pub. Date: Oct. 22, 2026

(54) ADULT ENDOCHONDRAL BONE GROWTH SYSTEM

(71) Applicant: Nolan Francis Chowansky, Bluffton, SC (US)

(72) Inventor: Nolan Francis Chowansky, Bluffton, SC (US)

(21) Appl. No.: 19/183,927

(22) Filed: Apr. 21, 2025

(51) Int. Cl. A61L 27/52; A61L 27/12; A61L 27/20; A61L 27/22

(52) U.S. Cl. CPC A61L 27/52; A61L 27/12; A61L 27/20; A61L 27/227; A61L 2430/02

(57) ABSTRACT

A multifunctional composition and method enhance skeletal structure in adults by modulating bone density and inducing controlled longitudinal elongation. The composition creates a micro-environmental growth zone at targeted sites in skeletally mature patients, mimicking an active epiphyseal plate for endochondral ossification. This enables precise bone elongation with synchronized growth of adjacent musculoskeletal tissues. Applicable to osteoporosis, skeletal dysplasias, joint conditions, and cosmetic limb lengthening, the system integrates regeneration with biomechanical adaptation via localized delivery.

### BACKGROUND OF THE INVENTION

### Field of the Invention

The present invention relates generally to bone tissue engineering and orthopedic regenerative medicine, and more specifically to minimally invasive methods and compositions for promoting bone lengthening in skeletally mature individuals. In particular, the invention concerns an injectable hydrogel formulation capable of reactivating endochondral growth processes in closed epiphyseal plates for the purpose of longitudinal bone elongation in adults.

### Description of Related Art

Current methods for bone lengthening in humans are largely based on surgical procedures, such as distraction osteogenesis. These methods require osteotomy (surgical cutting of the bone), followed by the application of external or internal mechanical devices to gradually separate the bone segments, stimulating new bone formation in the gap. For example, U.S. Pat. Nos. 5,626,581 and 6,673,079 B1 disclose mechanical devices for bone lengthening and reshaping by distraction osteogenesis. While effective, these approaches are invasive, painful, and associated with long recovery periods and risks of infection, non-union, and device-related complications.

In contrast, various hydrogel compositions have been explored for orthopedic applications, particularly for growth plate repair in pediatric patients and for localized bone regeneration. For instance, WO2017152112A2 and CN-111617318-A describe hydrogel systems aimed at regenerating damaged epiphyseal cartilage in children. These technologies are intended to support natural growth in immature skeletal systems and do not contemplate or enable growth plate reactivation in skeletally mature adults.

Other related art includes U.S. Pat. No. 4,467,809, which discloses a method for non-invasive electrical stimulation to enhance growth through open epiphyseal plates. However, such methods are inherently ineffective in adult patients, whose growth plates have undergone physiologic closure and ossification, typically by the end of puberty.

The prevailing consensus in medical and scientific literature is that once epiphyseal plates fuse during late adolescence, they cannot be reopened, thereby ending the body's natural capacity for longitudinal bone growth. This view is supported by clinical sources and scholarly publications, which confirm that closed growth plates ossify into solid bone and no known medical intervention exists to reverse this process.

To date, no known patent or publication discloses a minimally invasive, injectable hydrogel composition specifically formulated to reopen closed epiphyseal plates in adults for the purpose of bone lengthening. Prior art lacks any teaching or suggestion of a therapeutic method that combines injectable hydrogels, growth factors, stem cells, and osteoconductive materials to biologically simulate a temporary, functional growth plate in adult bone tissue.

Accordingly, there exists a long-felt but unsolved need for a less invasive, biologically integrative method for skeletal elongation in adults. The present invention addresses this need by introducing a novel, multifunctional hydrogel capable of creating a transient, regenerative growth zone within closed epiphyseal regions of mature bones. This approach enables safe, targeted, and controlled stimulation of longitudinal bone growth and density in adult patients, representing a significant advancement in the fields of orthopedic medicine, tissue engineering, and cosmetic height enhancement.

### SUMMARY OF THE INVENTION

The present invention introduces a multifunctional, injectable hydrogel system intended for systemic administration to improve bone density and promote controlled, micro-level longitudinal bone elongation in adult patients with physiologically closed epiphyseal plates. This invention distinctively facilitates the temporary reactivation of growth plate-like environments across multiple skeletal sites, enabling both therapeutic and cosmetic applications, including the treatment of bone density disorders, correction of skeletal dysplasias, and minimally invasive height enhancement.

In contrast to conventional site-specific bone therapies or surgical bone-lengthening methods, this invention utilizes a systemically delivered hydrogel that selectively targets skeletal structures. Upon administration, the hydrogel establishes biologically active microenvironments that trigger chondrogenesis and endochondral ossification within previously closed growth zones, leading to localized bone elongation, enhanced mineral density, and synchronized adaptation of surrounding soft tissues, all without requiring surgical osteotomy or mechanical distraction.

The hydrogel composition incorporates a biocompatible scaffold matrix composed of one or more hydrophilic or semi-synthetic polymers, such as hyaluronic acid, polyethylene glycol (PEG), collagen, alginate, chitosan, gelatin methacrylate (GelMA), or combinations thereof, but not limited to these. To enable the hydrogel to effectively function by opening the ossified epiphyseal plate through enzymatic degradation, the initial compound includes Matrix Metalloproteinase-13 (MMP-13) or similar compound. The matrix may also be modified with crosslinkable groups or functional moieties to enhance mechanical stability, injectability, or cellular interaction. To enable the hydrogel to effectively function by opening the ossified epiphyseal plate, the initial compound or method includes Matrix Metalloproteinase-13 (MMP-13), equivalent enzymes (e.g., collagenases, cathepsin K), chemical agents (e.g., EDTA, citric acid), or physical disruption (e.g., ultrasound, micro-drilling).

To support osteogenesis and bone remodeling, the formulation contains osteoconductive and osteoinductive nanoparticles or mineral-based additives, such as hydroxyapatite, bioactive glass, calcium phosphate (CaP), or other bioceramic particles known to promote bone mineralization and scaffold integration.

The hydrogel is embedded with one or more bioactive signaling molecules or growth factors, selected from Bone Morphogenetic Proteins (BMPs), Insulin-like Growth Factor 1 (IGF-1), Fibroblast Growth Factors (FGFs), Transforming Growth Factor-beta (TGF-β), Vascular Endothelial Growth Factor (VEGF), and other agents capable of stimulating chondrogenesis, angiogenesis, and endochondral ossification. These may be delivered in free form or encapsulated within microparticles or nanocarriers to enable sustained or sequential release profiles.

Optional biological components may include autologous or allogeneic mesenchymal stem cells (MSCs), induced pluripotent stem cells (iPSCs), or progenitor cells capable of differentiating into osteoblasts or chondrocytes in response to local cues. Hormonal modulators, such as parathyroid hormone analogs (e.g., teriparatide) or calcitonin, may also be included to further influence bone remodeling and mineralization processes. Peptide-based adhesion ligands, such as RGD sequences or matrix metalloproteinase-sensitive linkers, may be incorporated to facilitate cellular attachment and remodeling within the scaffold.

The formulation is designed for systemic or localized administration and is delivered using a triple-chamber or multi-reservoir system that allows for the separation and mixing of active components at the time of injection. Upon administration, the hydrogel undergoes in situ gelation, forming a spatially confined, bioactive matrix capable of promoting targeted skeletal tissue remodeling and biologically controlled bone elongation.

Specifically, the composition is engineered to create a transient, growth plate-like environment within previously ossified epiphyseal regions of adult bone. By reactivating localized chondrogenesis and supporting endochondral ossification, the hydrogel enables temporary reopening of the epiphyseal plate and controlled, self-limiting longitudinal bone growth. This mechanism of action provides a non-surgical alternative to traditional bone lengthening procedures and broadens the therapeutic window for skeletal modification in skeletally mature individuals.

This invention overcomes a long-standing limitation in adult skeletal biology by enabling the reactivation of closed growth zones and inducing biologically integrated bone lengthening without the need for surgical osteotomy, external hardware, or permanent implants. Furthermore, the composition enhances bone density and supports micro-level bone elongation in adult patients with closed epiphyseal plates. As a result, this system represents a major breakthrough in regenerative orthopedic therapy and minimally invasive skeletal enhancement, applicable to both medical and cosmetic purposes. It promotes bone density and micro-level bone elongation, allowing sufficient time for the skeletal structure to adapt and achieve the desired lengthening.

### BRIEF DESCRIPTION OF THE DRAWINGS

FIG. 1 illustrates an adult long bone with a closed epiphyseal line, and an adolescent long bone prior to epiphyseal plate closure.

FIG. 2 illustrates the delivery system, featuring a triple-chamber syringe with pre-loaded bioactive compartments. It also shows the localized injection of the multifunctional hydrogel at the epiphyseal line site, facilitating its opening.

FIG. 3 is a diagrammatic representation of the hydrogel's composite matrix, including max-degrading enzyme, scaffold materials, encapsulated growth factors, and nanoparticles.

### DETAILED DESCRIPTION OF THE INVENTION

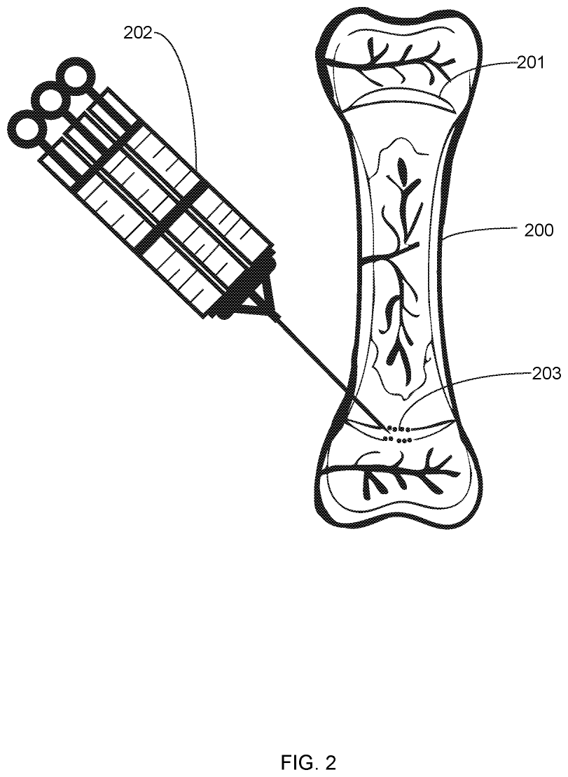

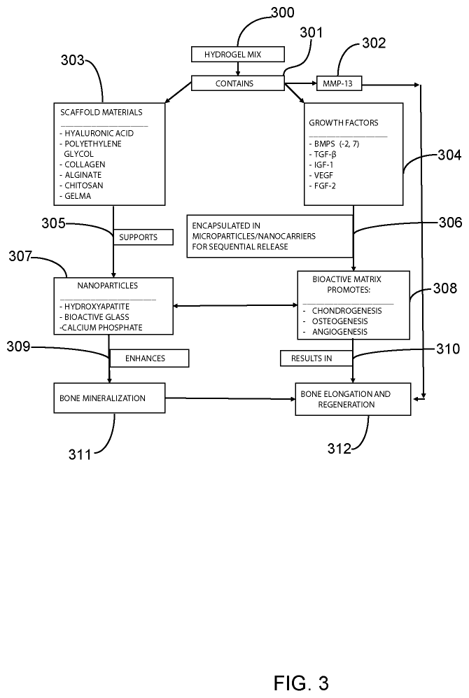

The present invention provides a multifunctional, injectable hydrogel system, termed the Adult Endochondral Bone Growth System, for enhancing bone density and inducing controlled, micro-level longitudinal bone growth in adult patients with closed epiphyseal plates. To overcome the challenge of mimicking an adolescent growth plate, the hydrogel combines bioactive cells, signaling molecules, mechanosensitive materials, and supportive therapies to create a synthetic growth zone that functionally emulates essential epiphyseal processes, as depicted in FIG. 2 (203). Although not fully replicating the intricate cellular, signaling, hormonal, and mechanical environment of a natural growth plate, this system facilitates controlled tissue growth and ossification, with FIG. 3 illustrating the bioactive matrix (300) promoting bone elongation (312). The hydrogel recreates a localized, growth plate-like environment through a minimally invasive procedure, initiating transient endochondral ossification for bone elongation while supporting adaptive responses in surrounding soft tissues, including tendons, ligaments, and muscles.

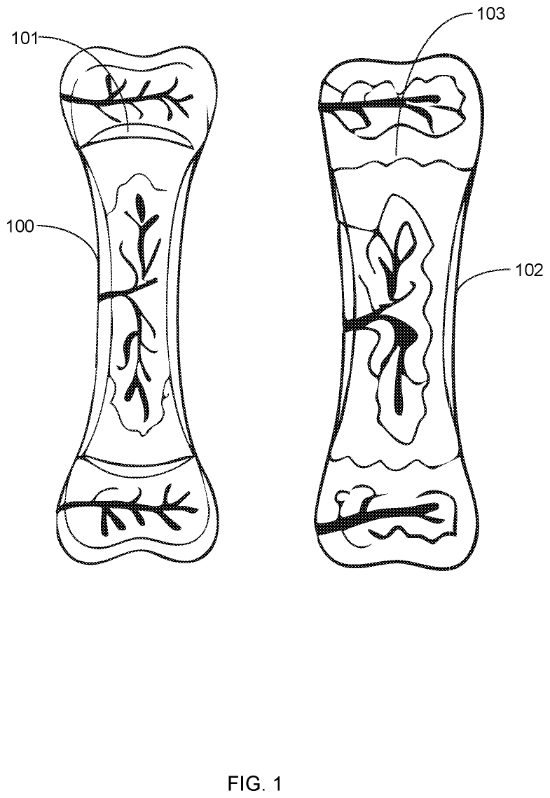

FIG. 1 illustrates a cross-sectional view of an adult bone (100) with a closed epiphyseal plate, showing the fused epiphyseal line (101), contrasted with an adolescent bone (102) exhibiting an open epiphyseal plate (103) and cartilaginous growth zone. This comparison highlights the challenge of inducing bone elongation in skeletally mature adults.

The hydrogel system comprises a biocompatible scaffold, matrix-degrading enzyme, bioactive agents, and nanoparticles delivered via a triple-chamber syringe to open the ossified epiphyseal line, initiate micro-growth, enhance bone density, and facilitate re-closure. The scaffold includes hydrophilic or semi-synthetic polymers, such as hyaluronic acid, polyethylene glycol (PEG), collagen, alginate, gelatin methacrylate (GelMA), chitosan, or combinations thereof. In preferred embodiments, an alginate-chitosan polyelectrolyte complex with tunable stiffness (2-10 kPa) promotes chondrogenic differentiation of mesenchymal stem cells, mimicking an active epiphyseal plate.

Hyaluronic acid provides a hydrophilic environment for cellular proliferation, differentiation, and nutrient diffusion. PEG enhances mechanical strength and viscoelasticity, ensuring injectability and structural stability. Collagen and GelMA mimic the extracellular matrix, promoting cellular adhesion, biochemical signaling, and host tissue integration. These scaffold materials establish a bioactive matrix for tissue formation post-epiphyseal opening.

The ossified epiphyseal line is softened using matrix metalloproteinase-13 (MMP-13) or equivalent enzymes, delivered via the first chamber of a triple-chamber syringe. MMP-13 degrades the calcified matrix, creating microchannels (1-2 mm depth) for cellular infiltration and hydrogel penetration. The second chamber releases scaffold materials (e.g., hyaluronic acid, PEG, collagen, alginate, GelMA, chitosan) with osteoconductive nanoparticles (e.g., hydroxyapatite, bioactive glass, calcium phosphate) and MMP-sensitive linkers to form an in situ gel, filling microchannels and providing a structural framework. The third chamber delivers growth factors (e.g., TGF-β, IGF-1, BMP-2, BMP-7, VEGF, FGF-2) and hormonal modulators (e.g., teriparatide) to stimulate chondrogenesis, osteogenesis, and localized bone resorption, preparing the site for cartilage formation.

The injection may create a micro-fracture at the epiphyseal line, enabling hydrogel infiltration. MMP-13 is encapsulated in fast-release liposomes or microparticles to act rapidly without degrading scaffold materials. Scaffold materials gel in situ, and growth factors are encapsulated in microparticles or nanocarriers for sequential release: TGF-β and IGF-1 promote early chondrogenesis, followed by BMP-2 for osteogenesis. This three-chamber system prevents premature interactions, ensuring controlled release and minimizing tissue damage via a single injection.

In alternative embodiments, pre-treatment or concurrent treatment with enzymes (e.g., MMP-13, collagenases), chemical agents (e.g., EDTA), biological remodeling agents (e.g., RANKL, teriparatide), or mechanical disruption (e.g., ultrasound, micro-drilling) may facilitate hydrogel injection, guided by imaging (e.g., CT, MRI).

However, the single-injection triple-chamber system is preferred for minimal invasiveness.

Osteoconductive nanoparticles (e.g., hydroxyapatite, bioactive glass, calcium phosphate) in the scaffold chamber promote bone mineralization, serving as nucleation sites for calcium deposition and mimicking natural bone matrix. Growth factors (e.g., BMP-2, BMP-7, TGF-β, IGF-1, VEGF, FGF-2) and hormonal modulators (e.g., teriparatide), housed in the third chamber, are released sequentially to induce chondrogenesis, cartilage deposition, and osteogenesis, preventing premature ossification.

Additional additives, such as RGD peptides, MMP-sensitive crosslinkers, or degradation-tunable linkers, may enhance scaffold integration and remodeling. Osteoinductive agents (e.g., silicate-based particles) may increase mineralization and synchronize scaffold resorption with tissue regeneration.

The hydrogel is pre-loaded into a triple-chamber syringe with a fine-gauge needle (18-25 gauge) or steerable catheter, guided by imaging (e.g., CT, MRI, fluoroscopy) for precise delivery. MMP-13 degrades the calcified matrix, scaffold materials form an in situ gel, and growth factors initiate biological activity. Gelation may occur via thermoresponsive polymers, pH shift, ionic exchange, or enzymatic crosslinking.

FIG. 2 depicts a cross-sectional view of an adult bone (200) during hydrogel administration via the triple-chamber syringe (202, 300), containing (301) MMP-13 (302), scaffold materials (303), and growth factors (304). The closed epiphyseal line (201) is opened (203) to create a growth plate-like environment, facilitating endochondral ossification and longitudinal growth.

The hydrogel establishes a microenvironment mimicking an active epiphyseal plate. Growth factors activate chondrogenesis and cartilage deposition, forming a synthetic growth zone. Endochondral ossification gradually replaces cartilage with bone, achieving incremental lengthening. The hydrogel degrades over 3-6 months, ceasing chondrogenic activity, while BMP-2 promotes terminal ossification, allowing epiphyseal re-closure.

Growth is monitored via periodic imaging (e.g., X-ray, MRI) or biomarkers (e.g., cartilage oligomeric matrix protein, type II collagen fragments). If excessive growth occurs, a quenching mechanism (e.g., calcitonin analog, BMP inhibitor like noggin) halts chondrogenesis, promoting ossification. The hydrogel's degradation profile may also ensure self-limited growth.

FIG. 3 is a block diagram of the hydrogel (300), comprising MMP-13 (302), scaffold materials (303) (e.g., hyaluronic acid, PEG, collagen, alginate, chitosan, GelMA), growth factors (304) (e.g., BMP-2, BMP-7, TGF-β, IGF-1, VEGF, FGF-2, teriparatide), and nanoparticles (307) (e.g., hydroxyapatite, bioactive glass, calcium phosphate). These form a bioactive matrix (308), promoting chondrogenesis, osteogenesis, and angiogenesis, enhancing (309) bone mineralization (311), and resulting in bone elongation and regeneration (312).

The hydrogel's biocompatible components minimize immune responses. Antimicrobial agents (e.g., silver nanoparticles, vancomycin-loaded microparticles) and immune modulators (e.g., dexamethasone, anti-inflammatory peptides) may be included to prevent infection and inflammation. Degradation products are non-toxic and metabolized naturally. Monitoring via imaging or biomarkers (e.g., C-reactive protein) detects adverse reactions.

The system treats conditions such as osteoporosis, osteogenesis imperfecta, Paget's disease, bone necrosis, and skeletal dysplasias, and supports structural deformity correction, spinal reinforcement, and joint stabilization. Cosmetically, it enables minimally invasive limb lengthening and height enhancement, applicable to any bone via a single injection.

Patient-specific factors (e.g., age, bone density, hormonal status) are assessed pre-treatment using dual-energy X-ray absorptiometry or blood tests. The hydrogel formulation may be adjusted (e.g., higher TGF-β for older patients, softer scaffold for osteoporotic bones) to ensure efficacy across diverse populations.

### Conclusion

The present invention provides a multifunctional hydrogel system for safely reopening closed epiphyseal plates in adults, using advanced biomaterials, growth factors, and optional cellular components to induce controlled, temporary endochondral ossification. This minimally invasive injectable formulation promotes micro-level bone elongation and density enhancement, addressing medical needs (e.g., osteoporosis, skeletal dysplasias) and cosmetic goals (e.g., limb lengthening) in regenerative orthopedics.

1. A multifunctional injectable hydrogel composition for inducing controlled longitudinal bone elongation and enhancing bone density in a skeletally mature adult patient having at least one physiologically closed epiphyseal plate, the composition comprising:
(a) a biocompatible scaffold matrix configured to form an injectable hydrogel in situ;
(b) a matrix-remodeling component configured to locally modify calcified tissue within the physiologically closed epiphyseal plate to facilitate formation of the localized temporary synthetic growth zone capable of supporting endochondral ossification;
(c) one or more osteoconductive nanoparticles or mineral-based additives;
(d) one or more bioactive signaling molecules or growth factors configured to promote chondrogenesis, osteogenesis, angiogenesis, and endochondral ossification;
(e) a delivery formulation configured to enable localized administration of the hydrogel composition to the physiologically closed epiphyseal plate;
(f) the hydrogel composition being configured, following localized administration, to undergo in situ gelation and establish a spatially confined bioactive matrix within the physiologically closed epiphyseal plate;
wherein the composition is configured, upon localized administration, to form a localized temporary synthetic growth zone within the physiologically closed epiphyseal plate that functionally emulates an active epiphyseal plate by promoting localized chondrogenesis followed by endochondral ossification, thereby producing biologically regulated longitudinal bone elongation.

2. The hydrogel composition of claim 1, wherein the biocompatible scaffold matrix comprises one or more hydrophilic or semi-synthetic polymers configured to establish the spatially confined bioactive matrix that supports formation of the localized temporary synthetic growth zone.

3. The hydrogel composition of claim 1, wherein the one or more bioactive signaling molecules or growth factors are configured to provide a temporally coordinated release profile that promotes localized chondrogenesis followed by endochondral ossification within the localized temporary synthetic growth zone.

4. The hydrogel composition of claim 3, wherein the one or more bioactive signaling molecules or growth factors are encapsulated to provide a sequential release profile in which a first release phase promotes localized chondrogenesis within the localized temporary synthetic growth zone and a subsequent release phase promotes endochondral ossification to produce biologically regulated longitudinal bone elongation.

5. The hydrogel composition of claim 1, further comprising one or more bioactive matrix-interactive components configured to facilitate localized cellular attachment, tissue remodeling, or integration within the spatially confined bioactive matrix to support formation and maturation of the localized temporary synthetic growth zone.

6. The hydrogel composition of claim 1, wherein the spatially confined bioactive matrix is configured to establish and maintain a transient growth plate-like microenvironment within the physiologically closed epiphyseal plate that supports localized chondrogenesis followed by endochondral ossification.

7. The hydrogel composition of claim 1, further comprising one or more biological remodeling modulators configured to regulate localized bone remodeling during formation and maturation of the localized temporary synthetic growth zone.

8. The hydrogel composition of claim 1, further comprising one or more progenitor or stem cell populations configured to cooperate with the one or more bioactive signaling molecules to promote localized chondrogenesis and endochondral ossification within the localized temporary synthetic growth zone.

9. The hydrogel composition of claim 1, wherein the composition is configured for administration using a multi-reservoir delivery system that separately maintains matrix-remodeling, scaffold-forming, and bioactive signaling components prior to administration and combines the components during localized delivery to establish the localized temporary synthetic growth zone within the physiologically closed epiphyseal plate.

10. The hydrogel composition of claim 1, wherein the localized temporary synthetic growth zone is configured to undergo formulation-controlled endochondral ossification followed by re-establishment of a non-proliferative epiphyseal state as the bioactive matrix degrades, thereby terminating longitudinal bone growth.

11. The hydrogel composition of claim 1, wherein the localized temporary synthetic growth zone is configured to transition from a regenerative state to a non-proliferative epiphyseal state through coordinated regulation of bioactive matrix degradation and biological signaling, thereby terminating longitudinal bone growth.

12. The hydrogel composition of claim 1, further comprising one or more optional bioactive components configured to cooperate with the hydrogel composition to regulate localized tissue remodeling, chondrogenesis, endochondral ossification, or angiogenesis during formation and maturation of the localized temporary synthetic growth zone.

13. A multifunctional injectable hydrogel composition for inducing controlled longitudinal bone elongation and enhancing bone density in a skeletally mature adult patient having at least one physiologically closed epiphyseal plate, the composition comprising:
(a) a biocompatible scaffold matrix configured to form an injectable hydrogel in situ;
(b) a matrix-remodeling component configured to locally modify calcified tissue within the physiologically closed epiphyseal plate to facilitate formation of a localized temporary synthetic growth zone capable of supporting endochondral ossification;
(c) one or more osteoconductive nanoparticles or mineral-based additives;
(d) one or more bioactive signaling molecules or growth factors configured to promote chondrogenesis, osteogenesis, angiogenesis, and endochondral ossification;
(e) a delivery formulation configured to enable localized administration of the hydrogel composition to the physiologically closed epiphyseal plate;
(f) the hydrogel composition being configured, following localized administration, to undergo in situ gelation and establish a spatially confined bioactive matrix within the physiologically closed epiphyseal plate;
wherein the composition is configured, upon localized administration, to form a localized temporary synthetic growth zone within the physiologically closed epiphyseal plate that functionally emulates an active epiphyseal plate by promoting localized chondrogenesis followed by endochondral ossification, thereby producing biologically regulated longitudinal bone elongation and enhanced bone density.

14. The hydrogel composition of claim 13, further comprising one or more optional bioactive components configured to cooperate with the hydrogel composition to regulate localized tissue remodeling, chondrogenesis, angiogenesis, and endochondral ossification during formation and maturation of the localized temporary synthetic growth zone.
